(19) United States
(12) Patent Application Publication
(10) Pub. No.: US 2026/0079572 A1
(43) Pub. Date: Mar. 19, 2026

(54) Method and System for Prompting User Abnormalities Based on Nystagmus Monitoring

(71) Applicant: Tongji Hospital affiliated to Tongji Medical college of Huazhong University of Science & Technology, Wuhan (CN)

(72) Inventors: Jing Wang, Wuhan (CN); Ming Zeng, Wuhan (CN)

(21) Appl. No.: 19/325,418

(22) Filed: Sep. 10, 2025

(30) Foreign Application Priority Data
Sep. 14, 2024 (CN) 202411291712.3

(51) Int. Cl. G06F 3/01; G06N 3/0475; G06V 40/18; G06V 40/20; G16H 50/20; G16H 50/30

(52) U.S. Cl. CPC G06F 3/013; G06N 3/0475; G06V 40/18; G06V 40/20; G16H 50/20; G16H 50/30

(57) ABSTRACT

Provided are a method and a system for prompting user abnormalities based on nystagmus monitoring, the method including: acquiring eyeball movement information about a user in real time via a nystagmus monitoring device, and generating an eyeball movement trajectory of the user; in the eyeball movement trajectory of the user, screening respective regular eyeball movement trajectories through a regular trajectory analysis strategy, and recognizing a nystagmus degree of each nystagmus type corresponding to the user through a nystagmus determination strategy; predicting an abnormality probability of the user and an abnormality time period of the user through an abnormality analysis network based on the nystagmus degree of the respective nystagmus types and the interval time between the respective regular eyeball movement trajectories, and performing an abnormality precaution prompt operation to the user through the nystagmus monitoring device, thereby improving the accuracy of predicting the user's abnormalities.

### CROSS REFERENCES TO RELATED APPLICATION

The present application claims the benefit of Chinese Patent Application No. 202411291712.3 filed on Sep. 14, 2024, the contents of which are incorporated herein by reference in their entirety.

### TECHNICAL FIELD

The present application relates to the technical field of nystagmus monitoring, and particularly relates to a method and system for prompting user abnormalities based on nystagmus monitoring.

### BACKGROUND ART

Nystagmus (NY) is an involuntary, rhythmic, back-and-forth swinging eyeball movement. Directions include horizontal, vertical, rotational, etc., among which the horizontal type is common. The direction of nystagmus is usually indicated by the direction of a fast phase, and the fast phase is the movement that compensatorily restores a fixation position. It is referred to as nystagmus. Frequent or intermittent nystagmus may cause user abnormalities such as dizziness, fainting, and shock. Therefore, improving real-time monitoring of nystagmus is a key focus for enhancing the prediction of user abnormalities.

The traditional method for predicting nystagmus involves testing at professional medical institutions, followed by expert analysis to estimate the approximate time when a user may experience abnormalities. However, this method cannot predict the user's nystagmus in real-time, resulting in low accuracy in predicting the user's abnormalities.

### SUMMARY OF THE INVENTION

A main objective of the present application is to provide a method and system for prompting user abnormalities based on nystagmus monitoring, which is intended to solve the problem in the prior art that the prediction accuracy of user abnormality is low due to the inability to predict the user's nystagmus in real time.

In order to achieve the above objective, the present application provides a method for prompting user abnormalities based on nystagmus monitoring, the method including:
acquiring eyeball movement information about a user in real time via a nystagmus monitoring device, and generating an eyeball movement trajectory of the user based on the eyeball movement information about the user;
in the eyeball movement trajectory of the user, screening respective regular eyeball movement trajectories through a regular trajectory analysis strategy, and recognizing a nystagmus degree of each nystagmus type corresponding to the user through a nystagmus determination strategy based on the respective regular eyeball movement trajectories;
predicting an abnormality probability of the user and an abnormality time period of the user through an abnormality analysis network based on the nystagmus degree of the respective nystagmus types and the interval time between the respective regular eyeball movement trajectories, and performing an abnormality precaution prompt operation to the user through the nystagmus monitoring device based on the abnormality probability of the user and the abnormality time period of the user.

In addition, in order to achieve the above objective, the present application further provides a system for prompting user abnormalities based on nystagmus monitoring, the system for prompting user abnormalities based on nystagmus monitoring including:
an acquisition module configured to acquire eyeball movement information about a user in real time via a nystagmus monitoring device, and generating an eyeball movement trajectory of the user based on the eyeball movement information about the user;
a recognition module configured to, in the eyeball movement trajectory of the user, screen respective regular eyeball movement trajectories through a regular trajectory analysis strategy, and recognize a nystagmus degree of each nystagmus type corresponding to the user through a nystagmus determination strategy based on the respective regular eyeball movement trajectories; and
a precaution module configured to predict an abnormality probability of the user and an abnormality time period of the user through an abnormality analysis network based on the nystagmus degree of the respective nystagmus types and the interval time between the respective regular eyeball movement trajectories, and perform an abnormality precaution prompt operation to the user through the nystagmus monitoring device based on the abnormality probability of the user and the abnormality time period of the user.

The present application provides a method and system for prompting user abnormalities based on nystagmus monitoring. Using a nystagmus monitoring device, eyeball movement information of the user is acquired in real time to analyze the regular eyeball movements estimated in the user's eyeball movement trajectory. The nystagmus degree for the nystagmus type the user may currently be experiencing is determined to analyze the user's abnormality probability and abnormality time period. This avoids the problems of low timeliness and inability to monitor in real time associated with nystagmus monitoring in medical institutions. At the same time, the user's abnormality probability and abnormality time period are predicted based on the nystagmus degree for the nystagmus type monitored in real time, which can effectively prevent additional harm to the human body caused by abnormalities due to nystagmus in the user, and can provide the user with time for preparation and precaution in advance, thereby greatly reducing the degree of additional harm caused by nystagmus to the user. In this solution, the user's eyeball movement data is acquired in real time, and the eyeball movement trajectory corresponding to the eyeball movement data is analyzed from multiple perspectives to predict the user's abnormalities and improve the prediction accuracy of the user's abnormalities.

### BRIEF DESCRIPTION OF THE DRAWINGS

To more clearly illustrate the solutions in the present application, a brief introduction will be given below to the accompanying drawings required in the description of the embodiments of the present application. Obviously, the accompanying drawings in the following description are some embodiments of the present application, and for a person skilled in the art, other accompanying drawings can also be obtained based on these drawings without making creative efforts.

FIG. 1 is a device structural diagram of a nystagmus monitoring device according to an embodiment of the present application.

FIG. 2 is a flowchart of a method for prompting user abnormalities based on nystagmus monitoring according to an embodiment of the present application.

FIG. 3 is a schematic structural diagram of a system for prompting user abnormalities based on nystagmus monitoring according to an embodiment of the present application.

FIG. 4 is an internal structural view of a computer device according to an embodiment of the present application.

### DETAILED DESCRIPTION OF THE INVENTION

The method for prompting user abnormalities based on nystagmus monitoring provided by the embodiments of the present application can be applied to an application environment of the nystagmus monitoring device. The nystagmus monitoring device includes a support unit, an eyeball image acquisition unit, an analysis and control unit, and a precaution prompt unit. The eyeball image acquisition unit, the analysis and control unit and the precaution prompt unit are arranged on the support unit, the eyeball image acquisition unit and the precaution prompt unit are respectively connected to the analysis and control unit. The analysis and control unit can be a terminal for control and analysis, and the terminal includes an operation chip and an instruction transceiving interface. The terminal acquires eyeball movement information of the user in real time to analyze the regular eyeball movements estimated in the user's eyeball movement trajectory, then, determines the nystagmus degree for the nystagmus type the user may currently be experiencing to analyze the user's abnormality probability and abnormality time period. This avoids the problems of low timeliness and inability to monitor in real time associated with nystagmus monitoring in medical institutions. At the same time, the user's abnormality probability and abnormality time period are predicted based on the nystagmus degree for the nystagmus type monitored in real time, which can effectively prevent additional harm to the human body caused by abnormalities due to nystagmus in the user, and can provide the user with time for preparation and precaution in advance, thereby greatly reducing the degree of additional harm caused by nystagmus to the user. In this solution, the user's eyeball movement data is acquired in real time, and the eyeball movement trajectory corresponding to the eyeball movement data is analyzed from multiple perspectives to predict the user's abnormalities and improve the prediction accuracy of the user's abnormalities.

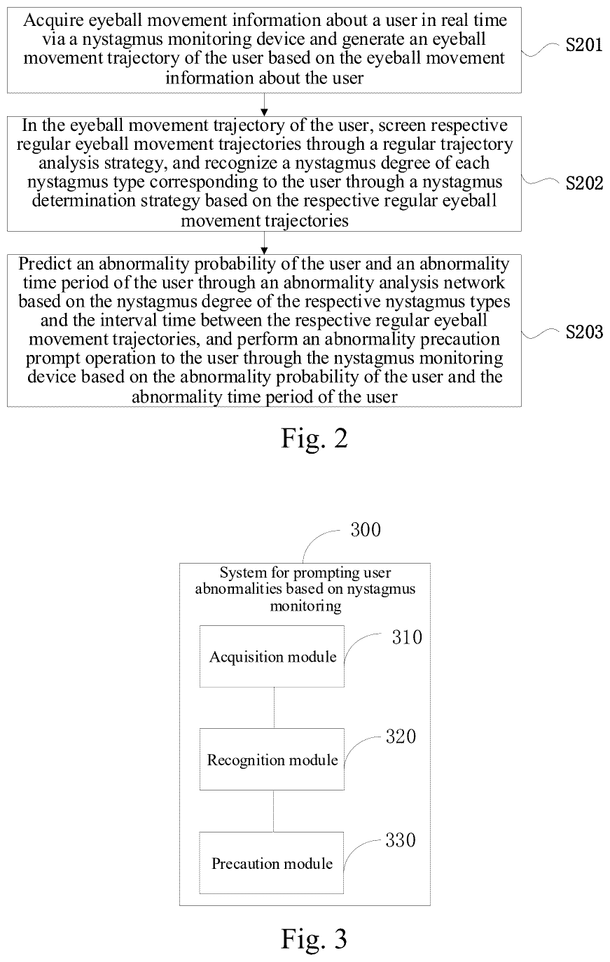

In one embodiment, as shown in FIG. 2, a method for prompting user abnormalities based on nystagmus monitoring is provided. Taking the application of this method to a terminal of the analysis and control unit as an example for explanation, the method includes the following steps: step S201 Acquire eyeball movement information about a user in real time via a nystagmus monitoring device, and generate an eyeball movement trajectory of the user based on the eyeball movement information about the user.

In the present embodiment, the terminal acquires the eyeball image information of the user in real time via the eyeball image acquisition unit of the nystagmus monitoring device, and takes the eyeball image information acquired at each moment as the eyeball movement information of the user. The eyeball image acquisition unit can be a single-frame camera device, and the single-frame camera device can acquire an image of an eyeball part of the user according to each frame. The terminal constructs the eyeball movement trajectory of the user based on the eyeball movement information of the user. The eyeball movement trajectory is an eyeball movement trajectory established in a three-dimensional coordinate system of the eyeball with the user's eye central point as the origin, the eyeball surface as a plane, and the time axis as a horizontal axis, where the unit of the time axis is a millisecond, and the unit of the eyeball surface is millimeter. That is, the eyeball movement trajectory includes an actual movement trajectory corresponding to a movement distance and a time feature of the actual movement trajectory. The specific generation process will be described in detail later.

Step S202 In the eyeball movement trajectory of the user, screen respective regular eyeball movement trajectories through a regular trajectory analysis strategy, and recognize a nystagmus degree of each nystagmus type corresponding to the user through a nystagmus determination strategy based on the respective regular eyeball movement trajectories.

In the present embodiment, the terminal screens respective regular eyeball movement trajectories through a regular trajectory analysis strategy in the eyeball movement trajectory of the use, and recognizes a nystagmus degree of each nystagmus type corresponding to the user through a nystagmus determination strategy based on the respective regular eyeball movement trajectories. The regular trajectory analysis strategy refers to the strategy of dividing the pupil movement information between the position information of the pupil central points in the eyeball image information at each moment into respective trajectory groups corresponding to different moment counts according to the incremental grouping operation of moment counts corresponding to different frame counts, and then recognizing respective similar trajectory groups of similar movement trajectories corresponding to each moment count. The specific recognition process will be described in detail later. The nystagmus determination strategy is a strategy for analyzing the regular eyeball movement trajectories of each similar trajectory group of each similar movement trajectory, so as to recognize the nystagmus type and nystagmus degree corresponding to each similar movement trajectory. The specific determination process will be described in detail later. Step S203 Predict an abnormality probability of the user and an abnormality time period of the user through an abnormality analysis network based on the nystagmus degree of the respective nystagmus types and the interval time between the respective regular eyeball movement trajectories, and perform an abnormality precaution prompt operation to the user through the nystagmus monitoring device based on the abnormality probability of the user and the abnormality time period of the user.

In the present embodiment, the terminal predicts the user's abnormality probability and abnormality time period through the abnormality analysis network based on the nystagmus degree for each nystagmus type and the interval time between respective regular eyeball movement trajectories. Then, based on the user's abnormality probability and abnormality time period, it performs an abnormality precaution prompt operation to the user through the nystagmus monitoring device. The user's abnormality probability is the likelihood that the user may experience an abnormality in the near future, and the abnormality time period is the time frame during which the user may experience an abnormality in the near future. Such abnormalities include, but are not limited to, syncope, vertigo, dizziness, coma, and other physical abnormalities caused by nystagmus. The specific prediction process will be described in detail later.

Based on the above scheme, by acquiring eyeball movement information of the user in real time, the regular eyeball movements estimated in the user's eyeball movement trajectory are analyzed. The nystagmus degree for the nystagmus type the user may currently be experiencing is determined to analyze the user's abnormality probability and abnormality time period. This avoids the problems of low timeliness and inability to monitor in real time associated with nystagmus monitoring in medical institutions. At the same time, the user's abnormality probability and abnormality time period are predicted based on the nystagmus degree for the nystagmus type monitored in real time, which can effectively prevent additional harm to the human body caused by abnormalities due to nystagmus in the user, and can provide the user with time for preparation and precaution in advance, thereby greatly reducing the degree of additional harm caused by nystagmus to the user. In this solution, the user's eyeball movement data is acquired in real time, and the eyeball movement trajectory corresponding to the eyeball movement data is analyzed from multiple perspectives to predict the user's abnormalities and improve the prediction accuracy of the user's abnormalities.

Alternatively, the generating an eyeball movement trajectory of the user based on the eyeball movement information about the user includes: the eyeball movement information about the user is split into eyeball image information at respective moments, and position information about a pupil central point in each piece of eyeball image information is recognized; position information about a pupil central point in the eyeball image information at adjacent moments is performed connection processing to obtain pupil movement information about the user at respective moments, and a three-dimensional coordinate system of the user's eyeball is constructed based on a range of the user's eyeball.

In the present embodiment, the terminal splits the eyeball movement information about the user into eyeball image information at respective moments, and recognizes position information about a pupil central point in each piece of eyeball image information. The position information of the pupil central point in each eyeball image is recognized by marking the position of the pupil central point in the eyeball image information via an image recognition network, and the position information corresponding to the position of the pupil central point is recognized in a two-dimensional coordinate system corresponding to the eyeball image information. The image recognition network is a convolutional neural network.

Then position information about a pupil central point in the eyeball image information at adjacent moments is performed connection processing to obtain pupil movement information about the user at respective moments, and a three-dimensional coordinate system of the user's eyeball is constructed based on a range of the user's eyeball. Subsequently, the terminal projects the pupil movement information between respective moments into the eyeball three-dimensional coordinate system to obtain the eyeball movement trajectory of the user. The trajectory between respective moments in the eyeball movement trajectory is pupil movement information containing a time feature. That is, the pupil movement information is a two-dimensional movement trajectory, and the eyeball movement trajectory is a three-dimensional movement trajectory.

Based on the above-mentioned scheme, the eyeball movement trajectory of the user is constructed by recognizing the change information of the pupil central point position at different moments, so that the practicality and accuracy of the constructed eyeball movement trajectory are improved. Alternatively, in an eyeball movement trajectory of a user, the screening respective regular eyeball movement trajectories through a regular trajectory analysis strategy: the eyeball movement trajectory of the user is divided into respective trajectory groups of each moment count according to respective moments, and for each moment count, an overlapping movement trajectory between sub-eyeball movement trajectories of every two trajectory groups of the moment count is recognized; a trajectory overlapping degree between every two trajectory groups is calculated by means of an overlapping degree algorithm based on an overlapping movement trajectory between sub-eyeball movement trajectories of every two trajectory groups, and a trajectory deviation value of every two trajectory groups is calculated by means of a trajectory deviation algorithm based on the pupil movement information between respective moments of every two trajectory groups; and based on the trajectory overlapping degree between every two trajectory groups and the trajectory deviation value between every two trajectory groups, each similar trajectory group of similar movement trajectories between the respective trajectory groups is screened, and the sub-eyeball movement trajectories corresponding to each similar trajectory group are taken as respective regular eyeball movement trajectories.

In the present embodiment, the terminal divides the eyeball movement trajectory of the user into respective trajectory groups of each moment count according to respective moments, and for each moment count, recognizes an overlapping movement trajectory between sub-eyeball movement trajectories of every two trajectory groups of the moment count. For each trajectory group of each moment count, for example, with five frame counts as the moment count, each sub-eyeball movement trajectory of adjacent five frame counts is each trajectory group of the moment count of 5 frame counts, and among them, each sub-eyeball movement trajectory of every five frame counts screened from the starting moment of each eyeball image information is the sub-eyeball movement trajectory of adjacent five frame counts. The moment count may be, but is not limited to, 2 frames, 5 frames, 10 frames, 15 frames, etc.

Then a trajectory overlapping degree between every two trajectory groups is calculated by means of an overlapping degree algorithm based on an overlapping movement trajectory between sub-eyeball movement trajectories of every two trajectory groups, and a trajectory deviation value of every two trajectory groups is calculated by means of a trajectory deviation algorithm based on the pupil movement information between respective moments of every two trajectory groups. The trajectory overlapping degree is a proportional value between the length of the overlapping movement trajectory between the two sub-eyeball movement trajectories and the length of each sub-eyeball movement trajectory after each starting time of the two sub-eyeball movement trajectories is set to be the same moment, namely, the trajectory overlapping degree between the two trajectory groups. The trajectory deviation value of the two trajectory groups is an average of the deviation values between the pupil movement information between respective moments of each of the two trajectory groups.

Based on the trajectory overlapping degree between every two trajectory groups and the trajectory deviation value between every two trajectory groups, the terminal screens each similar trajectory group of similar movement trajectories between the respective trajectory groups, and takes the sub-eyeball movement trajectories corresponding to each similar trajectory group as respective regular eyeball movement trajectories. Each similar trajectory group of similar movement trajectories has a trajectory overlapping degree between each similar trajectory group being greater than an overlapping degree threshold value pre-set at a terminal, and a trajectory deviation value being lower than a deviation value threshold value pre-set at a terminal. That is, each similar trajectory group of a plurality of similar movement trajectories may be included in the same moment count. Based on the above-mentioned scheme, through frame division and grouped analysis, the comprehensiveness and accuracy of recognizing each regular eyeball movement trajectory are improved.

Alternatively, the recognize a nystagmus degree of each nystagmus type corresponding to the user through a nystagmus determination strategy based on the respective regular eyeball movement trajectories includes: based on respective moments corresponding to the respective regular eyeball movement trajectories corresponding to each moment count, each regular eyeball movement trajectory is performed trajectory de-repetition processing to obtain each target regular eyeball movement trajectory, and for each similar movement trajectory, trajectory features corresponding to the similar movement trajectory are recognized based on each target regular eyeball movement trajectory corresponding to the similar movement trajectory; a trajectory type of the similar movement trajectory is recognized based on respective trajectory features corresponding to the similar movement trajectories, and a trajectory range of the similar movement trajectories is recognized based on each target regular eyeball movement trajectory corresponding to the similar movement trajectory; and a nystagmus type corresponding to each trajectory type is queried in a nystagmus database, and a nystagmus degree of the respective nystagmus types is recognized through a nystagmus degree evaluation strategy of the nystagmus type based on the trajectory range of the similar movement trajectories of respective trajectory types corresponding to each nystagmus type.

In the present embodiment, based on respective moments corresponding to the respective regular eyeball movement trajectories corresponding to each moment count, each regular eyeball movement trajectory is performed trajectory de-repetition processing to obtain each target regular eyeball movement trajectory, and for each similar movement trajectory, trajectory features corresponding to the similar movement trajectory are recognized based on each target regular eyeball movement trajectory corresponding to the similar movement trajectory. The deduplication processing method is as follows: in respective regular eyeball movement trajectories, if two regular eyeball movement trajectories have an inclusion relationship, that is, trajectory A completely includes trajectory B, or trajectory B is completely included in trajectory A, the terminal removes trajectory B and only retains trajectory A. Therefore, after deduplication, the number of target regular eyeball movement trajectories corresponding to each similar movement trajectory may remain unchanged or decrease. The method for recognizing a trajectory feature corresponding to a movement trajectory is to project respective regular eyeball movement trajectories onto an eyeball plane to obtain trajectory distribution information corresponding to respective regular eyeball movement trajectories, and then the terminal recognizes a trajectory shape feature corresponding to each piece of trajectory distribution information via an image feature recognition network, where the image feature recognition network is a convolutional neural network based on a self-attention mechanism.

The terminal recognizes a trajectory type of the similar movement trajectories based on respective trajectory features corresponding to the similar movement trajectories, and recognizes a trajectory range of the similar movement trajectories based on respective target regular eyeball movement trajectories corresponding to the similar movement trajectories. The trajectory database includes trajectory types corresponding to different trajectory features, and each trajectory type corresponds to one or more trajectory features. The terminal recognizes the trajectory types of the similar movement trajectories in the trajectory database based on the corresponding trajectory features of the similar movement trajectories. When all the trajectory features of the similar movement trajectory belong to the trajectory type, the terminal determines that the trajectory type is the trajectory type of the similar movement trajectory. The trajectory type may be, but is not limited to, a horizontal straight trajectory type, an elliptical trajectory type, a circular trajectory type, a vertical straight trajectory type, a horizontal curved trajectory type, a vertical curved trajectory type, and the like.

The terminal queries a nystagmus type corresponding to each trajectory type in a nystagmus database, and recognizes a nystagmus degree of the respective nystagmus types through a nystagmus degree evaluation strategy of the nystagmus type based on the trajectory range of the similar movement trajectories of respective trajectory types corresponding to each nystagmus type. The nystagmus database stores therein nystagmus types corresponding to different trajectory types, where each nystagmus type can correspond to one or more trajectory types, and each trajectory type can only correspond to one nystagmus type, and the nystagmus types include a horizontal type, a vertical type and a rotary type. The trajectory type corresponding to the horizontal type includes a horizontal straight-line trajectory type and a horizontal curved-line trajectory type; the trajectory types corresponding to the vertical type include a vertical curve trajectory type and a vertical straight-line trajectory type; the trajectory types corresponding to the rotation include an elliptical trajectory type and a circular trajectory type. Each trajectory range is, for example, a straight-line length of a horizontal straight-line trajectory type, a trajectory-diameter length of a circular trajectory type, a two-focus distance length of an elliptical trajectory type, etc. The nystagmus degree evaluation strategy for each nystagmus type includes nystagmus degree values corresponding to different standard trajectory ranges, and the terminal determines a sub-nystagmus degree of the nystagmus type corresponding to each similar movement trajectory based on the standard trajectory range to which the trajectory range of each similar movement trajectory belongs. The terminal calculates the average value of the sub-nystagmus degree for each nystagmus type as the nystagmus degree for each nystagmus type.

Based on the above-mentioned scheme, by screening the regular eyeball movement trajectories, the problem that similar movement estimation affects the accuracy of data calculation is avoided. By recognizing the types of the target regular eyeball movement trajectories and analyzing the nystagmus degree, the accuracy and comprehensiveness of the recognition are improved.

Alternatively, the predicting an abnormality probability of the user and an abnormality time period of the user through an abnormality analysis network based on the nystagmus degree of the respective nystagmus types and the interval time between the respective regular eyeball movement trajectories includes: for each similar movement trajectory, an interval time between respective target regular eyeball movement trajectories is recognized based on an interval moment range between respective target regular eyeball movement trajectories of the similar movement trajectory; a nystagmus frequency of the nystagmus type corresponding to the similar movement trajectory is calculated based on an interval time between respective target regular eyeball movement trajectories, and a nystagmus duration of the nystagmus type corresponding to the similar movement trajectory is recognized based on a moment range contained in all the target regular eyeball movement trajectories corresponding to the similar movement trajectory; and the abnormality probability of the user and an abnormality time period of the user are predicted through the abnormality analysis network based on a nystagmus degree of each nystagmus type, an average nystagmus frequency of each nystagmus type, and an average nystagmus duration of each nystagmus beauty type.

In the present embodiment, for each similar movement trajectory, the terminal recognizes an interval time between respective target regular eyeball movement trajectories based on an interval moment range between respective target regular eyeball movement trajectories of the similar movement trajectory. The terminal calculates a nystagmus frequency of the nystagmus type corresponding to the similar movement trajectory based on an interval time between respective target regular eyeball movement trajectories. The nystagmus frequency is the number of target regular eyeball movement trajectories per unit time interval range, and the unit time interval is a preset time interval for the terminal, for example, 60 frames, 120 frames, 180 frames, 240 frames, etc.

The terminal recognizes a nystagmus duration of the nystagmus type corresponding to the similar movement trajectory based on a moment range contained in all the target regular eyeball movement trajectories corresponding to the similar movement trajectory. The nystagmus duration is an average duration of the moment groups corresponding to each target regular eyeball movement trajectory corresponding to the similar movement trajectory. The terminal calculates the average value of the nystagmus duration for each nystagmus type, the average value of the nystagmus frequency for each nystagmus type, and obtains the average nystagmus frequency and the average nystagmus duration for each nystagmus type.

The terminal predicts the abnormality probability of the user and the abnormality time period of the user through the abnormality analysis network based on a nystagmus degree of each nystagmus type, an average nystagmus frequency of each nystagmus type, and an average nystagmus duration of each nystagmus beauty type. The abnormality analysis network is an artificial neural network based on a reinforcement learning neural network.

Based on the above scheme, by recognizing the nystagmus degree of each nystagmus type, the average nystagmus frequency of each nystagmus type, and the average nystagmus duration of each nystagmus type for a user, and then analyzing the user's abnormality probability and abnormality time period, the accuracy and comprehensiveness of the analysis are improved.

Alternatively, the perform an abnormality precaution prompt operation to the user through the nystagmus monitoring device based on the abnormality probability of the user and the abnormality time period of the user includes: prompt template information about the nystagmus monitoring device and a prompt advance duration of the nystagmus monitoring device are acquired; in a prompt database, target prompt information corresponding to an abnormality probability of the user is queried, and the target prompt information is filled into the prompt template information to obtain current prompt information about the user; a prompt time point of the current prompt information is determined based on the abnormality time period of the user and the prompt advance duration, and a prompt instruction of the nystagmus monitoring device is generated based on the prompt time point and the current prompt information; and the prompt instruction is sent to the nystagmus monitoring device, and the nystagmus monitoring device is controlled to transmit the current prompt information to the user at the prompt time point.

In the present embodiment, the terminal acquires prompt template information about the nystagmus monitoring device, and a prompt advance duration of the nystagmus monitoring device. The prompt template information is the template content of the prompt information, and the template content includes the filling positions for prompt information corresponding to different abnormality probabilities and other template prompt information. In a prompt database, the terminal queries target prompt information corresponding to an abnormality probability of the user, and fills the target prompt information into the prompt template information to obtain current prompt information about the user. The prompt database includes target prompt information corresponding to each abnormality probability range. The terminal recognizes the abnormality probability range to which the user's abnormality probability belongs, so as to determine the target prompt information corresponding to the user's abnormality probability. The prompt information may include text information, vibration count information, vibration frequency information, buzz count information, buzz frequency information, etc.

The terminal determines a prompt time point of the current prompt information based on the abnormality time period of the user and the prompt advance duration, and generates a prompt instruction of the nystagmus monitoring device based on the prompt time point and the current prompt information. The terminal sends the prompt instruction to the precaution prompt unit of the nystagmus monitoring device, and controls the precaution prompt unit of the nystagmus monitoring device to transmit the current prompt information to the user at the prompt time point. Based on the above-mentioned scheme, by generating different current prompt information based on different abnormality probabilities, the comprehensiveness of the prompt information and the accuracy of the prompt are improved.

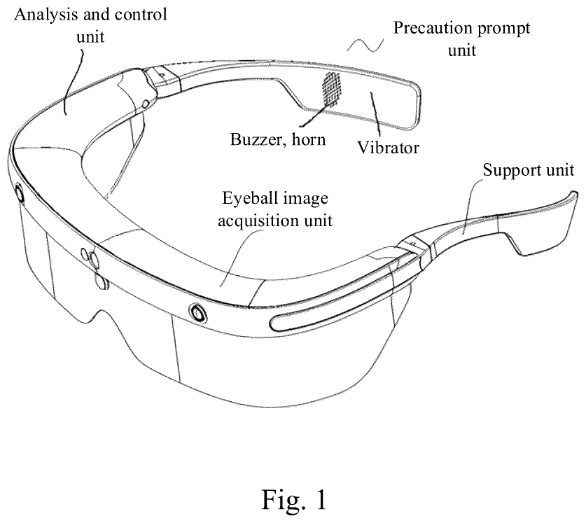

In one embodiment, as shown in FIG. 1, a nystagmus monitoring device is provided, the device including a support unit, an eyeball image acquisition unit, an analysis and control unit, and a precaution prompt unit, where:
the eyeball image acquisition unit, the analysis and control unit and the precaution prompt unit are arranged on the support unit, the eyeball image acquisition unit and the precaution prompt unit are respectively connected to the analysis and control unit; the support unit may be, among other things, an eyeglass holder.

The support unit is arranged at an outer side of the eyes of a user, and is used for arranging the eyeball image acquisition unit directly in front of the outer side of the eyeball, and arranging the precaution prompt unit at an ear contour of the user. That is to say, the eyeball image acquisition unit is arranged directly above the frame of the eye support and faces towards the eyeball part of the user, and the precaution prompt unit is arranged at the temple of the eye support.

The eyeball image acquisition unit is configured to acquire eyeball movement information about the user in real time, and send the eyeball movement information to the analysis and control unit. The eyeball image acquisition unit can be a single-frame camera device provided on a support unit. The analysis and control unit is configured to generate an eyeball movement trajectory of the user based on the eyeball movement information about the user when the eyeball movement information is received; in the eyeball movement trajectory of the user, screen respective regular eyeball movement trajectories through a regular trajectory analysis strategy, and recognize a nystagmus degree of each nystagmus type corresponding to the user through a nystagmus determination strategy based on the respective regular eyeball movement trajectories; predict an abnormality probability of the user and an abnormality time period of the user through an abnormality analysis network based on the nystagmus degree of the respective nystagmus types and the interval time between the respective regular eyeball movement trajectories, and generate a prompt instruction based on the abnormality probability of the user and the abnormality time period of the user; and send the prompt instruction to the precaution prompt unit. The analysis and control unit includes, in addition to an operation chip and an instruction transceiving interface, an information transceiving unit. When operations such as a neural network, image recognition, image feature extraction, etc. are involved, the analysis and control unit sends data involved in the above-mentioned operations and recognition information corresponding to an operation mode needing to be operated to a remote server corresponding to the device so as to perform the operations via the remote server. After the operations of the remote server are completed, the operation result is fed back to the analysis and control unit, so as to avoid the problem of low processing efficiency of the analysis and control unit. It improves the efficiency of real-time monitoring and real-time early warning.

The precaution prompt unit includes a timing module and a prompt module, and after receiving the prompt instruction, a current time point is detected via the timing module; and when the current time point is a prompt time point, transmitting the current prompt information to the prompt module, and transmitting the current prompt information to the user via the prompt module. The timing module includes a timer and an information transmission apparatus. The prompt module includes a small loudspeaker for transmitting prompt information about the text content; a buzzer used for executing buzz count information and buzz frequency information in current prompt information; the vibrator is used for executing the vibration count information and the vibration frequency information in the current prompt information.

It should be understood that although the respective steps in the flowcharts involved in the above-mentioned embodiments are displayed in sequence as indicated by the arrows, these steps are not necessarily executed in the sequence indicated by the arrows. Unless explicitly stated herein, there is no strict order restriction on the execution of these steps, and these steps can be executed in other orders. Moreover, at least some of the steps in the flowcharts involved in the above-mentioned embodiments may include multiple steps or multiple stages, which are not necessarily completed at the same moment, but can be executed at different moments, and the execution order of these steps or stages is not necessarily sequential, but can be executed alternately or alternately with at least some of other steps or steps or stages in other steps.

Based on the same inventive concept, embodiments of the present application further provide a system for prompting user abnormalities based on nystagmus monitoring for implementing the method for prompting user abnormalities based on nystagmus monitoring mentioned above. The solution provided by the system to solve the problem is similar to that described in the above-mentioned method. Therefore, the specific definitions in one or more embodiments of the system for prompting user abnormalities based on nystagmus monitoring provided below can refer to the definitions of the method for prompting user abnormalities based on nystagmus monitoring mentioned above, and will not be repeated here.

Further referring to FIG. 3, as an implementation of the method shown in FIG. 1 above, the present application provides an embodiment of a system 300 for prompting user abnormalities based on nystagmus monitoring, which includes an acquisition module 310, a recognition module 320, and a precaution module 330, where:
an acquisition module 310 is configured to acquire eyeball movement information about a user in real time via a nystagmus monitoring device, and generating an eyeball movement trajectory of the user based on the eyeball movement information about the user;
a recognition module 320 is configured to, in the eyeball movement trajectory of the user, screen respective regular eyeball movement trajectories through a regular trajectory analysis strategy, and recognize a nystagmus degree of each nystagmus type corresponding to the user through a nystagmus determination strategy based on the respective regular eyeball movement trajectories; and
a precaution module 330 is configured to predict an abnormality probability of the user and an abnormality time period of the user through an abnormality analysis network based on the nystagmus degree of the respective nystagmus types and the interval time between the respective regular eyeball movement trajectories, and perform an abnormality precaution prompt operation to the user through the nystagmus monitoring device based on the abnormality probability of the user and the abnormality time period of the user.

Alternatively, the acquisition module 310 is specifically configured to:
split the eyeball movement information about the user into eyeball image information at respective moments, and recognizing position information about a pupil central point in each piece of eyeball image information;
perform connection processing on position information about a pupil central point in the eyeball image information at adjacent moments to obtain pupil movement information about the user at respective moments, and construct a three-dimensional coordinate system of the user's eyeball based on a range of the user's eyeball; and
project the pupil movement information between respective moments into the eyeball three-dimensional coordinate system to obtain the eyeball movement trajectory of the user.

Alternatively, the recognition module 320 is specifically configured to:
divide the eyeball movement trajectory of the user into respective trajectory groups of each moment count according to respective moments, and for each moment count, recognize an overlapping movement trajectory between sub-eyeball movement trajectories of every two trajectory groups of the moment count;
calculate a trajectory overlapping degree between every two trajectory groups by means of an overlapping degree algorithm based on an overlapping movement trajectory between sub-eyeball movement trajectories of every two trajectory groups, and calculate a trajectory deviation value of every two trajectory groups by means of a trajectory deviation algorithm based on the pupil movement information between respective moments of every two trajectory groups; and
based on the trajectory overlapping degree between every two trajectory groups and the trajectory deviation value between every two trajectory groups, screen each similar trajectory group of similar movement trajectories between the respective trajectory groups, and take the sub-eyeball movement trajectories corresponding to each similar trajectory group as respective regular eyeball movement trajectories.

Alternatively, the recognition module 320 is specifically configured to:
based on respective moments corresponding to the respective regular eyeball movement trajectories corresponding to each moment count, perform trajectory de-repetition processing on each regular eyeball movement trajectory to obtain respective target regular eyeball movement trajectories, and for each similar movement trajectory, recognize trajectory features corresponding to the similar movement trajectories based on respective target regular eyeball movement trajectories corresponding to the similar movement trajectories;
recognize a trajectory type of the similar movement trajectories based on respective trajectory features corresponding to the similar movement trajectories, and recognize a trajectory range of the similar movement trajectories based on respective target regular eyeball movement trajectories corresponding to the similar movement trajectories; and
query a nystagmus type corresponding to each trajectory type in a nystagmus database, and recognize a nystagmus degree of the respective nystagmus types through a nystagmus degree evaluation strategy of the nystagmus type based on the trajectory range of the similar movement trajectories of respective trajectory types corresponding to each nystagmus type.

Alternatively, the precaution module 330 is specifically configured to:
for each similar movement trajectory, recognize an interval time between respective target regular eyeball movement trajectories based on an interval moment range between respective target regular eyeball movement trajectories of the similar movement trajectory;
calculate a nystagmus frequency of the nystagmus type corresponding to the similar movement trajectory based on an interval time between respective target regular eyeball movement trajectories, and recognize a nystagmus duration of the nystagmus type corresponding to the similar movement trajectory based on a moment range contained in all the target regular eyeball movement trajectories corresponding to the similar movement trajectory; and
predict the abnormality probability of the user and the abnormality time period of the user through the abnormality analysis network based on a nystagmus degree of each nystagmus type, an average nystagmus frequency of each nystagmus type, and an average nystagmus duration of each nystagmus beauty type.

Alternatively, the precaution module 330 is specifically configured to:
acquire prompt template information about the nystagmus monitoring device, and a prompt advance duration of the nystagmus monitoring device;
in a prompt database, query target prompt information corresponding to an abnormality probability of the user, and fill the target prompt information into the prompt template information to obtain current prompt information about the user;
determine a prompt time point of the current prompt information based on the abnormality time period of the user and the prompt advance duration, and generate a prompt instruction of the nystagmus monitoring device based on the prompt time point and the current prompt information; and
send the prompt instruction to the nystagmus monitoring device, and control the nystagmus monitoring device to transmit the current prompt information to the user at the prompt time point.

The respective modules of the system for prompting user abnormalities based on nystagmus monitoring described above may be implemented in whole or in part by software, hardware, or a combination thereof. The modules may be embedded in hardware or separate from the processor in the computer device, or may be stored in software in a memory in the computer device, such that the processor invokes operations corresponding to the modules.

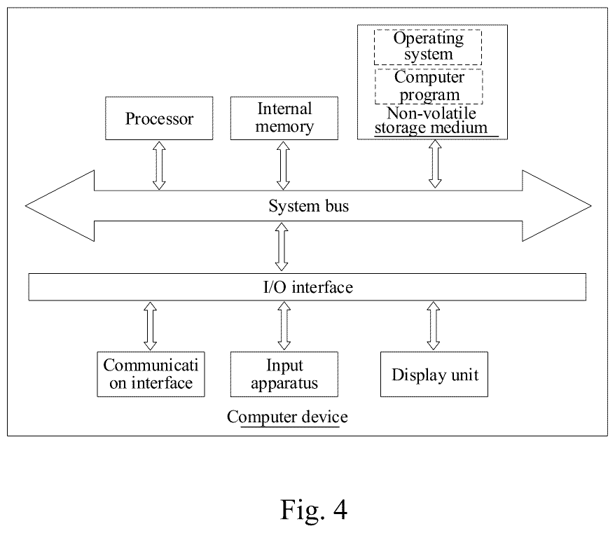

In one embodiment, a computer device is provided, which may be a terminal, the internal structure of which may be as shown in FIG. 4.

A person skilled in the art will appreciate that the structure illustrated in FIG. 4 is merely a block diagram of a portion of the structure related to the solution of the present application and does not limit the computer device to which the solution of the present application is applied. A specific computer device may include more or fewer components than those shown in the figure, or combine certain components, or have a different arrangement of components.

In one embodiment, a computer device is provided, which includes a memory and a processor, the memory storing a computer program which when executed by the processor carries out the steps of the method according to any one of the first aspects.

In one embodiment, a computer-readable storage medium is provided, on which a computer program is stored, which when executed by a processor, performs the steps of the method for prompting user abnormalities based on nystagmus monitoring as described above.

In one embodiment, a computer program product is provided, which includes a computer program, which when executed by a processor, implements the steps of the method for prompting user abnormalities based on nystagmus monitoring as described above.

The above embodiments only represent several implementation modes of the present application, and their descriptions are relatively specific and detailed, but they should not be construed as limiting the patent scope of the present application. It should be pointed out that for a person skilled in the art, several modifications and improvements can be made without departing from the concept of the present application, and these all fall within the protection scope of the present application. Therefore, the protection scope of the present application shall be subject to the appended claims.

1. A method for prompting user abnormalities based on nystagmus monitoring, comprising:
acquiring eyeball movement information about a user in real time via a nystagmus monitoring device, and generating an eyeball movement trajectory of the user based on the eyeball movement information about the user;
in the eyeball movement trajectory of the user, screening respective regular eyeball movement trajectories through a regular trajectory analysis strategy, and recognizing a nystagmus degree of each nystagmus type corresponding to the user through a nystagmus determination strategy based on the respective regular eyeball movement trajectories;
predicting an abnormality probability of the user and an abnormality time period of the user through an abnormality analysis network based on the nystagmus degree of the respective nystagmus types and the interval time between the respective regular eyeball movement trajectories, and performing an abnormality precaution prompt operation to the user through the nystagmus monitoring device based on the abnormality probability of the user and the abnormality time period of the user.

2. The method according to claim 1, wherein the generating an eyeball movement trajectory of the user based on the eyeball movement information about the user comprises:
splitting the eyeball movement information about the user into eyeball image information at respective moments, and recognizing position information about a pupil central point in each piece of eyeball image information;
performing connection processing on position information about a pupil central point in the eyeball image information at adjacent moments to obtain pupil movement information about the user at respective moments, and constructing a three-dimensional coordinate system of the user's eyeball based on a range of the user′ s eyeball; and
projecting the pupil movement information between respective moments into the eyeball three-dimensional coordinate system to obtain the eyeball movement trajectory of the user.

3. The method according to claim 2, wherein the in the eyeball movement trajectory of the user, screening respective regular eyeball movement trajectories through a regular trajectory analysis strategy comprises:
dividing the eyeball movement trajectory of the user into respective trajectory groups of each moment count according to respective moments, and for each moment count, recognizing an overlapping movement trajectory between sub-eyeball movement trajectories of every two trajectory groups of the moment count;
calculating a trajectory overlapping degree between every two trajectory groups by means of an overlapping degree algorithm based on an overlapping movement trajectory between sub-eyeball movement trajectories of every two trajectory groups, and calculating a trajectory deviation value of every two trajectory groups by means of a trajectory deviation algorithm based on the pupil movement information between respective moments of every two trajectory groups; and
based on the trajectory overlapping degree between every two trajectory groups and the trajectory deviation value between every two trajectory groups, screening each similar trajectory group of similar movement trajectories between the respective trajectory groups, and taking the sub-eyeball movement trajectories corresponding to each similar trajectory group as respective regular eyeball movement trajectories.

4. The method according to claim 3, wherein the recognizing a nystagmus degree of each nystagmus type corresponding to the user through a nystagmus determination strategy based on the respective regular eyeball movement trajectories comprises:
based on respective moments corresponding to the respective regular eyeball movement trajectories corresponding to each moment count, performing trajectory de-repetition processing on each regular eyeball movement trajectory to obtain respective target regular eyeball movement trajectories, and for each similar movement trajectory, recognizing trajectory features corresponding to the similar movement trajectories based on respective target regular eyeball movement trajectories corresponding to the similar movement trajectories;
recognizing a trajectory type of the similar movement trajectories based on respective trajectory features corresponding to the similar movement trajectories, and recognizing a trajectory range of the similar movement trajectories based on respective target regular eyeball movement trajectories corresponding to the similar movement trajectories; and
querying a nystagmus type corresponding to each trajectory type in a nystagmus database, and recognizing a nystagmus degree of the respective nystagmus types through a nystagmus degree evaluation strategy of the nystagmus type based on the trajectory range of the similar movement trajectories of respective trajectory types corresponding to each nystagmus type.

5. The method according to claim 4, wherein the predicting an abnormality probability of the user and an abnormality time period of the user through an abnormality analysis network based on the nystagmus degree of the respective nystagmus types and the interval time between the respective regular eyeball movement trajectories comprises:
for each similar movement trajectory, recognizing an interval time between respective target regular eyeball movement trajectories based on an interval moment range between respective target regular eyeball movement trajectories of the similar movement trajectory;
calculating a nystagmus frequency of the nystagmus type corresponding to the similar movement trajectory based on an interval time between respective target regular eyeball movement trajectories, and recognizing a nystagmus duration of the nystagmus type corresponding to the similar movement trajectory based on a moment range contained in all the target regular eyeball movement trajectories corresponding to the similar movement trajectory; and
predicting the abnormality probability of the user and the abnormality time period of the user through the abnormality analysis network based on a nystagmus degree of each nystagmus type, an average nystagmus frequency of each nystagmus type, and an average nystagmus duration of each nystagmus beauty type.

6. The method according to claim 5, wherein the performing an abnormality precaution prompt operation to the user through the nystagmus monitoring device based on the abnormality probability of the user and the abnormality time period of the user comprises:
acquiring prompt template information about the nystagmus monitoring device, and a prompt advance duration of the nystagmus monitoring device;
in a prompt database, querying target prompt information corresponding to an abnormality probability of the user, and filling the target prompt information into the prompt template information to obtain current prompt information about the user;
determining a prompt time point of the current prompt information based on the abnormality time period of the user and the prompt advance duration, and generating a prompt instruction of the nystagmus monitoring device based on the prompt time point and the current prompt information; and
sending the prompt instruction to the nystagmus monitoring device, and controlling the nystagmus monitoring device to transmit the current prompt information to the user at the prompt time point.

7. A nystagmus monitoring device, comprising a support unit, an eyeball image acquisition unit, an analysis and control unit, and a precaution prompt unit, wherein
the eyeball image acquisition unit, the analysis and control unit and the precaution prompt unit are arranged on the support unit, the eyeball image acquisition unit and the precaution prompt unit are respectively connected to the analysis and control unit;
the support unit is arranged at an outer side of the eyes of a user, and is used for arranging the eyeball image acquisition unit directly in front of the outer side of the eyeball, and arranging the precaution prompt unit at an ear contour of the user;
the eyeball image acquisition unit is configured to acquire eyeball movement information about the user in real time, and send the eyeball movement information to the analysis and control unit;
the analysis and control unit is configured to generate an eyeball movement trajectory of the user based on the eyeball movement information about the user when the eyeball movement information is received; in the eyeball movement trajectory of the user, screen respective regular eyeball movement trajectories through a regular trajectory analysis strategy, and recognize a nystagmus degree of each nystagmus type corresponding to the user through a nystagmus determination strategy based on the respective regular eyeball movement trajectories; predict an abnormality probability of the user and an abnormality time period of the user through an abnormality analysis network based on the nystagmus degree of the respective nystagmus types and the interval time between the respective regular eyeball movement trajectories, and generate a prompt instruction based on the abnormality probability of the user and the abnormality time period of the user; and send the prompt instruction to the precaution prompt unit;
the precaution prompt unit comprises a timing module and a prompt module, and after receiving the prompt instruction, a current time point is detected via the timing module; and when the current time point is a prompt time point, transmitting the current prompt information to the prompt module, and transmitting the current prompt information to the user via the prompt module.

8. A system for prompting user abnormalities based on nystagmus monitoring, comprising:
an acquisition module configured to acquire eyeball movement information about a user in real time via a nystagmus monitoring device, and generating an eyeball movement trajectory of the user based on the eyeball movement information about the user;
a recognition module configured to, in the eyeball movement trajectory of the user, screen respective regular eyeball movement trajectories through a regular trajectory analysis strategy, and recognize a nystagmus degree of each nystagmus type corresponding to the user through a nystagmus determination strategy based on the respective regular eyeball movement trajectories; and
a precaution module configured to predict an abnormality probability of the user and an abnormality time period of the user through an abnormality analysis network based on the nystagmus degree of the respective nystagmus types and the interval time between the respective regular eyeball movement trajectories, and perform an abnormality precaution prompt operation to the user through the nystagmus monitoring device based on the abnormality probability of the user and the abnormality time period of the user.

9. A computer device, comprising a memory and a processor, the memory storing a computer program, wherein the processor, when executing the computer program, implements the steps of the method according to claim 1.

10. A computer device, comprising a memory and a processor, the memory storing a computer program, wherein the processor, when executing the computer program, implements the steps of the method according to claim 2.

11. A computer device, comprising a memory and a processor, the memory storing a computer program, wherein the processor, when executing the computer program, implements the steps of the method according to claim 3.

12. A computer device, comprising a memory and a processor, the memory storing a computer program, wherein the processor, when executing the computer program, implements the steps of the method according to claim 4.

13. A computer device, comprising a memory and a processor, the memory storing a computer program, wherein the processor, when executing the computer program, implements the steps of the method according to claim 5.

14. A computer device, comprising a memory and a processor, the memory storing a computer program, wherein the processor, when executing the computer program, implements the steps of the method according to claim 6.

15. A non-transitory computer-readable storage medium having stored thereon a computer program, wherein the computer program, when executed by a processor, implements the steps of the method according to claim 1.

16. A non-transitory computer-readable storage medium having stored thereon a computer program, wherein the computer program, when executed by a processor, implements the steps of the method according to claim 2.

17. A non-transitory computer-readable storage medium having stored thereon a computer program, wherein the computer program, when executed by a processor, implements the steps of the method according to claim 3.

18. A non-transitory computer-readable storage medium having stored thereon a computer program, wherein the computer program, when executed by a processor, implements the steps of the method according to claim 4.

19. A non-transitory computer-readable storage medium having stored thereon a computer program, wherein the computer program, when executed by a processor, implements the steps of the method according to claim 5.

20. A non-transitory computer-readable storage medium having stored thereon a computer program, wherein the computer program, when executed by a processor, implements the steps of the method according to claim 6.
